(19) United States
(12) Patent Application Publication
(10) Pub. No.: US 2004/0044013 A1
(43) Pub. Date: Mar. 4, 2004

(54) 1-dimercaptoalkylquinazolin-2,4(1h,3h)-diones as matrix metalloproteinase (mmp) inhibitors

(76) Inventors: Jochen Heinicke, Leipzig (DE); Uwe Klausmeier, Guetersloh (DE); Christoph Arkona, Leipzig (DE); Siegfried Leistner, Leipzig (DE)

Correspondence Address: BURNS DOANE SWECKER & MATHIS L L P, POST OFFICE BOX 1404, ALEXANDRIA, VA, 22313-1404, US

(21) Appl. No.: 10/250,988

(22) PCT Filed: Dec. 20, 2001
(86) PCT No.: PCT/EP01/15170

(30) Foreign Application Priority Data
Jan. 13, 2001 (DE) 10101324.8

(51) Int. Cl. A61K 31/517; C07D 239/72

(52) U.S. Cl. 514/266.3; 544/285

(57) ABSTRACT

It is the object of this invention to discover and study new chemical substances of non-proteinogenic structure which have a matrix metalloproteinase (MMP)-inhibitory effect.

The object is achieved by the synthesis of dimercaptoalkyl-substituted quinazoline-2,4(1H,3H)diones.

Compounds of this substance class show a surprisingly clear and thus pharmacologically interesting MMP-inhibitory effect.

This invention relates to dimercaptoalkyl-substituted quinazoline-2,4(1H,3H)diones of general formula I

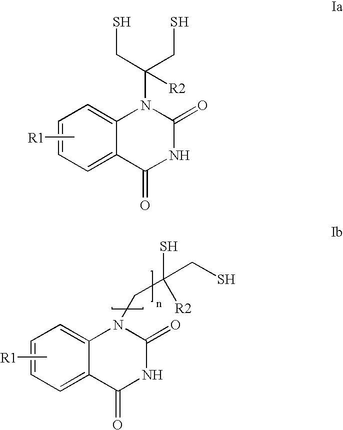

wherein:

R1=hydrogen, methyl, trifluoromethyl, methoxy, dimethoxy, Hal (Hal=fluorine, chlorine, bromine, iodine), hydroxy, carboxy

R2=hydrogen, methyl

n=1,2

and the tautomers and salts thereof as well as to the use as matrix metalloproteinase (MMP) inhibitors.

Quinazolines, quinazolinones and quinazolinediones are the subject of intensive pharmaceutical research. Their suitability as active substances and synthesis building blocks is not disputed.

The therapeutic effectiveness of sulfur-substituted quinazolinediones, particularly of 3(mercaptoalkyl)quinazoline-2,4(1H,3H)diones as pharmaceuticals was discovered by Leistner et al. (DD 298 784). It was found that there is immunostimulatory and antiviral effectiveness of representatives of this group of substances.

Little is currently known about the synthesis, and nothing is known about the effects, of dimercaptoalkyl-substituted quinazoline-2,4(1H,3H)diones.

Compounds having a dimercaptoalkyl substituent at the N1 nitrogen of the quinazoline-2,4(1H,3H)dione system corresponding to general formulae I have not yet been described in the technical literature.

Intensive research is being made in the field concerning the development of efficient, low-molecular and non-proteinogenic MMP inhibitors world-wide. It is known that from the physiological view-point the enzymatic activity of MMPs is subject to a strict, coordinated regulation between activation and inhibition. For this purpose, the organism has special proteins, what is called the tissue inhibitors of matrix metalloproteinases (TIMPs), which can inhibit rapidly and efficiently the activity of MMPs (Nagase, H. et al.: Engineering of selective TIMPs, 1-11; In: Inhibition of Matrix Metalloproteinases—Therapeutic Application (Eds. Greenwald, R. A., Zucker, S., Golub, L. M.) Ann NY Acad Sci 878 (1999). In particular in the case of the rheumatic diseases, an unblocked enzymatic activity of these enzymes results in the degradation of the cartilage substance and in chronic and painful changes of the joints, which is pathologically significant (Goldbach-Mansky, R. et al.: Active synovial matrix metalloproteinase-2 is associated with radiographic erosions in patients with early synovitis. Arthritis Res 2 (2000) 145-153).

The invasion and spread of tumors represents another example of the pathological effect of MMPs. Released and activated MMPs force their way through the dense collagenic connective tissue and in particular also through the basal membrane of the vessels, thus making it possible for the cancer cells to leave the tumor aggregation, migrate into the vessel system and form metastases elsewhere. MMPs also play a decisive role for the blood vessel supply of the growing tumor by forcing the way for the newly formed blood vessels through the collagenic connective tissue, thus being responsible for this vascularization of the growing tumor (Shapiro, S.D.: Matrix metalloproteinase degradation of extracellular matrix: biological consequences. Current Opinion in Cell Biology 10 (1998) 602-608).

The U.V.-induced erythema is to be mentioned as another relevant medical result of an inadequately slowed-down effect of MMPs. It occurs inter alia as a result of intensive solar radiation. The high-energy U.V. rays of sunlight or of tanning devices activate inter alia the inactive procollagenases in the irradiated skin, which as a consequence cleave collagen of the connective tissue and the blood capillaries, thus being responsible for the symptoms of a sunburn.

With these illustrative pathological effects of the unrestrained enzymatic MPP action, the consequences thereof may be prevented, or be reduced substantially, by stable MMP inhibitors. It is fascinating to realize the idea of inhibiting these enzymes in well-calculated fashion by specific inhibitors to thus stop e.g. a progressive cartilage destruction occurring in connection with a disease of the rheumatic form or prevent the growth or spread of tumors.

Numerous methods of obtaining compounds having an MMP-inhibiting effect are already known. These first generation active substances usually have a proteinogenic structure and are structurally related to natural inhibitors which are special proteins. These proteinogenic or pseudo-proteinogenic substrate analogues have as a structural element a zinc-binding group chelating the zinc ion in the active MMP center.

As to a therapeutic application, all such proteinogenic and non-proteinogenic active substances have a number of drawbacks, such as insufficient absorbability, usually short half-lives, only little stability as well as often undesired side-effects (Inhibition of Matrix Metalloproteinases Therapeutic Application (Eds. Greenwald, R. A., Zucker, S., Golub, L. M., Ann NY Acad Sci 878 (1999)).

Further developments made in this field yielded e.g. phosphonamide inhibitors, piperazine inhibitors, sulfonamide inhibitors, carbamate inhibitors, diazepine inhibitors, tetracycline inhibitors and, last but not least, hydroxamate inhibitors (Skotnicki, J. S. et al.: Design and synthetic considerations of matrix metalloproteinase inhibitors, 6172. In: Inhibition of Matrix Metalloproteinases—Therapeutic Application (Eds. Greenwald, R. A., Zucker, S., Golub, L. M.) Ann NY Acad Sci 878 (1999)). Although most of these developed inhibitors have impressive in vitro inhibitory effects and specificities, they showed a number of serious drawbacks in animal experiments and in humans when used in vivo. Here, cytotoxic reactions to a plurality of cells, a poor bioavailability and undesired side-effects, in particular a negative influence on the locomotor apparatus were to the fore.

Therefore, there is an urgent need for medicaments having a non-proteinogenic structure, which do not have the drawbacks of the active substances available thus far. In particular, there is a demand for new active substances which have an MMP-inhibitory effect, adequate stability and good absorbability, better pharmacokinetic properties and above all no undesired side-effects and cytotoxic reactions.

It is thus the object of the present invention to discover new chemical substances of non-proteinogenic structure which display an MMP-inhibitory effect. It is a further object of this invention to provide methods of producing such compounds and corresponding medicaments which contain said compounds.

This object is achieved according to the claims.

The inventive compounds, produced for the first time, of general formulae Ia and Ib belonging to the class of dimercaptoalkyl-substituted quinazoline-2,4(1H,3H)-diones show surprising, marked and thus pharmacologically interesting MMP-inhibitory effects which cannot be derived from formerly known relationships between structure and effect.

The effectiveness of the compounds according to the invention is proved by testing these inhibitors corresponding to general formulae Ia and Ib using different human MMPs (MMP-2, recombinant catalytic domain of MMP-3, MMP-8, MMP-9, recombinant catalytic domain of MMP-14).

The inventive dimercaptanes of general formulae I are obtained from the corresponding, angularly fused, partial hydrogenated thiazolo[3,2-a]quinazolinones or [1,3]thiazino-[3,2-a]quinazolinones of general formulae IIa, IIb or IIc

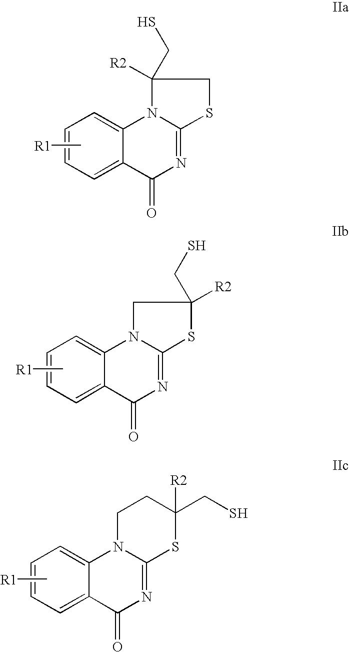

wherein:

R1=hydrogen, methyl, trifluoromethyl, methoxy, dimethoxy, Hal (Hal=fluorine, chlorine, bromine, iodine), carboxy

R2=hydrogen, methyl

by acid-catalyzed or base-catalyzed hydrolysis.

The tricyclic mercaptanes of general formulae II are obtained by reacting the corresponding tricyclic halogen precursors of general formulae III

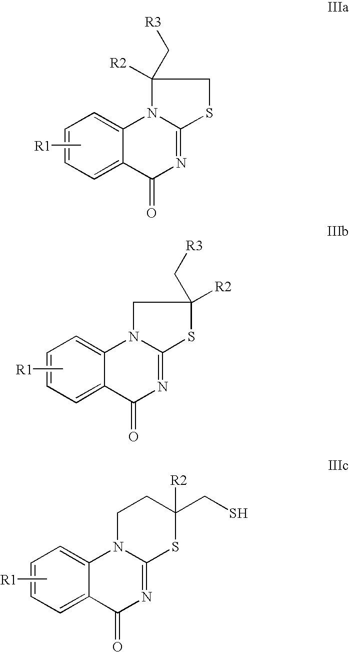

wherein:

R1=hydrogen, methyl, trifluoromethyl, methoxy, dimethoxy, Hal (Hal=fluorine, chlorine, bromine, iodine), carboxy

R2=hydrogen, methyl

R3=chlorine, bromine, iodine, tosyl

following the reaction with a sulfur-transmitting agent, preferably thiourea, in an inert solvent and subsequent mild saponification of the isolated intermediate stages, preferably of the isothiuronium salts.

The precursors of general formulae III are known in the literature (Leistner et al.).

### EXAMPLE 1

Preparation of

1-(1′,3′-Dimercapto-prop-2′-yl)quinazoline-2,4(1H,3H)dione (general formula Ia, R1=R2=hydrogen)

a.) (R,S)-1-Mercaptomethyl-1,2-dihydro-5H-thiazolo[3,2-a]quinazoline-5-one

(general formula IIa, R1=R2=hydrogen)

10 mmol of the (R,S)-1-bromomethyl-1,2-dihydro-5-thiazolo[3,2-b]quinazoline-5-one compound known in the literature (general formula IIIa, R1=R2=hydrogen, R3=bromine) are added to 30 ml monomethylglycol and heated with 12 mmol thiourea under TLC and/or HPLC control at a bath temperature of about 100° C. until the reaction is complete. If while cooling down no crystallization occurs, acetic ester will be added until turbidity appears and the then precipitating isothiuronium salt will be separated. The latter is saponified in 1 N NaOH by the addition of 10% (v/v) ethanol and under a nitrogen atmosphere at a bath temperature of 50° C. The course of the reaction is followed by means of TLC or HPLC chromatography. When the reaction is complete, acidification is effected with glacial acetic acid while ice cooling is carried out, the precipitate forming is sucked off, washed with water, dried and, when required, recrystallized from ethanol or acetic ester/heptane.

Colorless Crystals

F (fixed point): 157-61° C. (EtOH)

Yield: 35%

C₁₁H₁₀N₂OS₂ (250)

MS m/e (% B): M+250 (45), 203 (100)

b.) 1-(1′,3′-Dimercapto-prop-2′-yl)quinazoline-2,4(1H,3H)dione

(general formula Ia, R1=R2=hydrogen)

5 mmol of 1-mercaptomethyl-1,2-dihydro-5H-thiazolo[3,2-a]quinazoline-5-one obtained according to a.) are added to an acid mixture made of 0.6 ml concentrated sulfuric acid, 1.5 ml glacial acetic acid and 66 ml water and boiled to reflux under chromatographic control up to the complete conversion thereof. The precipitate obtained following cooling down (possibly dilute with water) will be separated, washed with water and recrystallized following drying.

Colorless Crystals

F.: 190-95° C.

Yield: 40%

C₁₁H₁₂N₂O₂S₂ (268)

IR (KBr, υ in cm⁻¹): 2558 (SH), 1687 (C═O), 1607 (C═O)

MS m/e (%B): 268 M+(5%), 221 (15%), 178 (25%), 163 (100

### EXAMPLE 2

(R,S)-1-(21,31-Dimercapto-prop-1′-yl)quinazoline-2,4(1H,3H)dione

(formula Ib, R1=R2=hydrogen, n=1)

a.) (R,S)-2-Mercaptomethyl-1,2-dihydro-5H-thiazolo[3,2-a]quinazoline-5-one

(general formula IIb, R1=R2=hydrogen)

The compound is obtained in analogy to Example 1, part a) (with methylglycol/i-propanol/DMF 1:1:2 as a solvent for the isothiuronium salt synthesis) from the (R,S)-2-bromomethyl-1,2-dihydro-5H-thiazolo[3,2-a]quinazoline-5-one compound known in the literature (general formula IIIb, R1=R2=hydrogen, R3=bromine).

Colorless Crystals

F.: 145-50° C.

Yield: 63%

C₁₁H₁₀N₂OS₂ (250)

MS m/e (%B): M+250 (48%), 203 (100%), 178 (85%)

b) (R,S)-1-(2′,3′-Dimercapto-prop-1′-yl)quinazoline-2,4(1H,3H)-dione

(formula Ib, R1=R2=hydrogen, n=1)

The compound is obtained in analogy to Example 1, part b) from (R,S)-2-mercaptomethyl-1,2-dihydro-5-thiazolo[3,2-a]quinazoline-5-one by acidic hydrolysis.

Colorless Crystals

F.: 147-51° C.

Yield: 83%

C₁₁H₁₂N₂O₂S₂ (268)

IR (KBr, υ in cm⁻¹): 2547 (SH), 1696 (C═O), 1609 (C═O)

MS m/e (%B): 268 M+(10%), 235 (60%), 201 (30%), 1633 (40%), 132 (100%)

### EXAMPLE 3

(R,S)-1-(3′,4′-Dimercapto-but-1′-yl)quinazoline-2,4(1H,3H)dione

(general formula Ib, R1=R2=hydrogen, n=2)

a.) (R,S)-3-mercaptomethyl-2,3-dihydro-1H,6H[1,3]thiazino[3,2-a]quinazoline-6-one (general formula IIc, R1=R2=hydrogen)

The compound is obtained in analogy to Example 2, part a) (the isothiuronium salt was precipitated using i-propanol) from the (R,S)-3-bromomethyl-2,3-dihydro-1H,6H-[1,3]-thiazino[3,2-a]quinazoline-6-one compound known in the literature (general formula IIIc, R1=R2=hydrogen, R3=bromine).

Colorless Crystals

F: 172-75° C.

Yield: 46%

C₁₂H₁₂N₂OS₂ (264)

MS m/e (%B): M+264 (35%), 231 (100%), 199 (30%)

b) (R,S)-1-(3′,4′-Dimercapto-but-1′-yl)quinazoline-2,4(1H,3H)dione

(general formula Ib, R1=R2=hydrogen, n=2)

The compound is obtained in analogy to Example 1, part b) from (R,S)-3-mercaptomethyl-2,3-dihydro-1H,6H-[1,3]-thiazino[3,2-a]quinazoline-6-one by acidic hydrolysis.

Colorless Crystals

F.: 125-28° C.

Yield: 40%

C₁₂H₁₄N₂O₂S₂ (282)

IR (KBr, U in cm⁻¹): 2553 (SH), 1700 (C═O), 1606 (C═O)

MS m/e (%B): 282): M+282 (5%), 249 (45%), 215 (35%), 163 (100%)

The MMP-inhibitory effect of the substances is determined as follows:

Inhibition of Matrix Metalloproteinase-2 (MMP-2)

MMP-2 (gelatinase) is supplied by cultured dermal fibroblasts to the culture medium in considerable amounts and is thus easily accessible. The secreted and inactive proform of the enzyme can be converted into the enzymatically active form by trypsin activation or by treatment with organic mercury compounds.

For this purpose, human dermal fibroblasts are obtained according to established standard methods and cultured and the cell-free culture supernatant is treated with trypsin. Trypsin is then inactivated with a specific inhibitor (TLCK) and active MMP-2 is partially purified by affinity chromatography on gelatin sepharose and subsequent gel filtration on sepharose. MMP-2 was identified and characterized by the availability of a commercial immunoassay.

Cloning and Expression of the Catalytic Domains of MT1-MMP and MMP-3

Based on the good experience with the use of the catalytic domain of MMP-8 in the kinetic measurements, two more recombinant expressions were established analogously, i.e. that of human membrane type 1 matrix metalloproteinase (MT1-MMP; MMP-14) and that of human MMP-3 (stromelysin-1). Both enzymes are exemplary representatives of subgroups of human matrix metalloproteinases. Both proteins may be renatured stably and in catalytically active form from inclusion bodies of transformed Escherichia coli cells. As in the recombinant MMP-8 thus the subsequent activation of the enzymes is dropped, which improves the reproducibility of the kinetic measurements. The purity of the recombinant enzyme domains was at least 70%.

The cloning and expression of the catalytic domains oriented itself by the following publications: Lichte A., Kolkenbrock H., Tschesche H.: The recombinant catalytic domain of membrane-type matrix metalloproteinase-1 (MT1-MMP) induces activation of progelatinase A and progelatinase A complexed with TIMP-2. FEBS Lett (1996), 397, 277-282 and Ye Q., Johnson L L, Hupe D J, Baragi V: Purification and characterization of the human stromelysin catalytic domain expressed in Escherichia coli, Biochemistry (1992), 31, 11231-11235.

Cloning and Expression of the Catalytic MMP-8 Domain

The catalytic MMP-8 domain was used as another test enzyme because it has a high degree of stability and is also available as an active enzyme and therefore needs not be activated, which is in consideration as one of the most frequent causes of error since the mercury compounds used for the activation often interfere with the test system and/or the enzyme and in this way can falsify the measuring results. The cloning strategy was directed towards the circumstance that instead of the whole enzyme only its enzymatically active catalytic domain was cloned into E. coli. By means of the constructed catalytic MMP-8 domain a stable, enzymatically active and highly pure enzyme was obtained which was very well suited for the routine tests of the inhibitory activity of the synthesized inhibitors and the measuring results of the individual series of measurements were absolutely comparable.

The cloning and expression of the recombinant catalytic MMP-8 domain was carried out in accordance with the information given by SCHNIERER et al. (Schnierer S., Kleine T., Gote T., Hillemann A., Knauper V., Tschesche H.: The recombinant catalytic domain of human neutrophil collagenase lacks type I collagen substrate specificity. Biochem Biophys Res Comm (1993), vol. 191, No. 2, 319-326).

Preparation of the Human Collagenase MMP-9

Native MMP-9 was obtained reproducibly and with good yield and purity from human Buffy Coat. For this purpose, Buffy Coat is brought to a final concentration of 0.4% using 10% (v/v) triton X-100, shaken on ice for 30 min. and then 1 vol. double binding buffer (40 mM Tris-HCl, pH 7.5, 10 mM CaCl₂, 1 M NaCl, 0.2% (v/v) triton X-100) is added and shaking on ice is continued for another 30 min.

The solution is centrifuged off at 16,000 rpm on an SS-34 rotor at 4° C. for 15 min. and filtered on glass wool. The filtrate is batched with gelatin agarose equilibrated with binding buffer and shaken on ice for 1 hour.

The charged gelatin agarose is transferred to a column and rinsed in protein-free manner using at least 10 vol. binding buffer. The bound MMP-9 is eluted with 2 gel volumes binding buffer plus 5% (v/v) DMSO.

For an exchange of buffers and simultaneous separation of minor MMP-2 contaminations the eluate can be separated on sephadex G-75 by means of gel filtration. Buffer I (20 mM Tris-HCl, pH 7.5, 5 mM CaCl₂, 100 mM NaCl, 0.1% (v/v) triton X-100) is used for this purpose.

The resulting eluate contains MMP-9 in the three known configurations: monomer, homodimer, heterodimer. The purity of the enzyme is about 90%, the rest of the foreign proteins being fibronectin and extremely small amounts of TIMPs.

The latent enzyme is activated by incubation at 37° C. using 1/100 vol. trypsin (10 mg/ml) for 30-60 min. Trypsin is inhibited by adding 1 mM PMSF or with a specific inhibitor (TLCK).

Quantitative Fluorescence Assay for Matrix Metalloproteinases

The fluorescent group Mca is separated from the internal quencher Dpa by enzymatic cleavage of the synthetic substrate Mca-Pro-Leu-Gly-Leu-Dpa-Ala-Arg-NH₂ using the respective collagenase. This is accompanied by a great increase in fluorescence in the measuring batch, which can be quantified using a fluorimeter (λₑₓ 328 nm, λₑₘ 393 nm) and proceeds linearly within the first few minutes. A certain specificity of the test to matrix metalloproteinases follows from the amino acid sequence -Pro-Leu-Gly-Leu- in the substrate, on the one hand, and from the select incubation conditions, on the other hand. Matrix metalloproteinases cleave the substrate at the Gly-Leu bond. The proteolytic residual activity of pre-incubated batches of enzyme and inhibitor is measured, the substrate and enzyme concentrations having been kept constant and the inhibitor concentration having been varied. Three series of measurements using a different substrate concentration were made for each tested inhibitor.

Assay

1984 μl measuring buffer (100 mM Tris-HCl, pH 7.5, 100 mM NaCl, 10 mM CaCl₂, 0.05% brij 35)

2 Al inhibitor dissolved in DMSO, or DMSO alone (non-inhibitory approach)

4 μl enzyme (MMP-2 or MMP-9 or catalytic domain of MMP-8)

5 min. preliminary incubation of the enzyme-inhibitor mixture at room temperature while stirring

start of the reaction with 10 μl substrate dissolved in DMSO

recording of the time-sensitive fluorescence increase over a period of 2 min.

Inhibition of Human MMPs by the Inhibitors of General Formulae Ia and Ib According to the invention

| C mpound | | | | | |
|---|---|---|---|---|---|
| according | | | | | |
| to Example | MMP-2 | MMP-3 | MMP-8 | MMP-9 | MT1-MMP |
| 1 | 50% | 50% | 70% | 60% | 30% inhi- |
| | inhibition | inhibition | inhibition | inhibition | bition with |
| | with 10 μM | with 10 μM | with 10 μM | with 10 μM | 10 μM |
| 2 | 75% | 75% | 75% | 80% | 60% |
| | inhibition | inhibition | inhibition | inhibition | inhibition |
| | with 4 μM | with 4 μM | with 4 μM | with 4 μM | with 4 μM |
| 3 | 50% | 50% | 50% | 50% | 250% |
| | inhibition | inhibition | inhibition | inhibition | inhibition |
| | with 5 μM | with 9 μM | with 3.5 μM | with 3.5 μM | with 4.5 μM |

1. Dimercaptoalkyl-substituted quinazoline-2,4-(1H,3H)diones of general formulae Ia and Ib 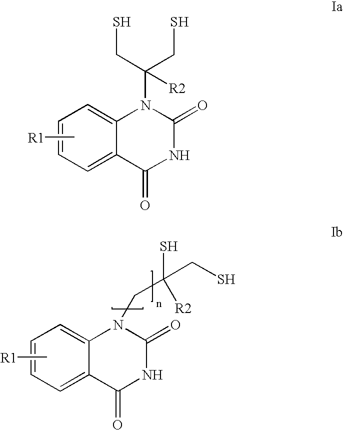
wherein
R1=hydrogen, methyl, methoxy, dimethoxy, Hal (Hal=fluorine, chlorine, bromine), hydroxy, carboxy,
R2=hydrogen, methyl
n=1,2
and the tautomers and salts thereof.

2. A method of producing compounds of formula Ia,
characterized in that
a given amount of the corresponding starting compound IIIa is added to monomethylglycol and heated with thiourea under TLC or HPLC control at a bath temperature of about 100° C. up to the full conversion thereof,
the precipitating isothiuronium salt is separated,
saponified in 1 N NaOH by adding ethanol at a bath temperature of about 50° C.,
admixed with glacial acetic acid while cooling,
the precipitate formed is separated and washed with water in known manner and recrystallized from ethanol,
the resulting substance is added to an acid mixture consisting of sulfuric acid, glacial acetic acid and water,
the mixture is heated up to the full conversion thereof,
the precipitate formed after cooling down is separated and purified in a generally known manner.

3. The method of producing compounds of formula Ib
characterized in that
a given amount of the corresponding starting compound IIIb is heated with thiourea in methylglycol/i-propanol/DMF 1:1:2 as solvent in a boiling water bath,
the resulting isothiuronium salt is separated following cooling,
saponified in 1 N NaOH by adding ethanol at a bath temperature of about 50° C., IIb forming,
which is hydrolyzed acidically and
following cooling the precipitate consisting of Ib is separated from the solvent.

4. Use of compounds of formula Ia as matrix metalloproteinase inhibitors.

5. Use of compounds of formula Ib as matrix metalloproteinase inhibitors.

6. Use of compounds of formula Ia as MMP inhibitors according to claim 4,
characterized in that
1-(1′,3′-dimercapto-prop-2′-yl)quinazoline-2,4(1H,3H)dione is used.

7. Use of compounds of formula IIa as MMP inhibitors according to claim 5,
characterized in that
(R,S) 1-(2′,3′-dimercapto-prop-1′-yl)-quinazoline-2,4(1H,3H)dione and/or (R,S)-1-(3′,4′-dimercapto-but-1′yl)quinazoline-2,4(1H,3H)dione are used.
